(19) United States
(12) Patent Application Publication
(10) Pub. No.: US 2021/0052384 A1
(43) Pub. Date: Feb. 25, 2021

(54) A MEDICAL FASTENING DEVICE

(71) Applicant: MEDTENTIA INTERNATIONAL LTD OY, Espoo (FI)

(72) Inventor: Olli Keränen, Bjarred (FI)

(21) Appl. No.: 16/960,070

(22) PCT Filed: Jan. 4, 2019
(86) PCT No.: PCT/FI2019/050003

§ 371 Date: Jul. 3, 2020

(30) Foreign Application Priority Data
Jan. 5, 2018 (FI) 20185010
Jan. 5, 2018 (FI) 20185011

(51) Int. Cl. A61F 2/24

(52) U.S. Cl. CPC A61F 2/2445; A61F 2230/0091; A61F 2220/0016

(57) ABSTRACT

A medical fastening device (100) for fastening or coupling an object (400A, 400B) into a tissue (20) have proximal (100A) and distal (100B) portions. It also comprises a helical body structure (107), where the helical body structure comprises a thread or ridge (104) having a lead (105) between said proximal and distal portions. The helical body structure comprises a first portion (101/103) and a second portion (102) next to said first portion (101). The thread of the helical body structure advantageously extends through the first (101) and second (102) portions and the properties, such as a pitch and/or lead, of the thread vary along the longitudinal axis (106) of the helical body structure so that the property of the thread has a first value in the first portion (101) and a second value in the second portion (102) so to provide different compressive force to the object and/or tissue introduced in said first and second portions of the helical body structure, and wherein the first value differs from the second value.

### TECHNICAL FIELD OF THE INVENTION

The invention relates to a medical fastening device for fastening an object to a tissue. In particularly the invention relates to a medical fastening device, which is suitable for fastening a cardiac implant (for example annuloplasty ring) into an annulus of a heart valve, for example a mitral valve or tricuspid valve, comprised of valve tissue and including the annulus and a plurality of leaflets. The principle of the invention can be applied for a catheter-operated or cannula-operated medical fastening device, but also an open-heart operated medical fastening device. Furthermore, the principle of the invention can be applied as well as to fastening also other object, such as tissue, implants or skin transplant beneath the skin or the like. In addition, the invention can also be used for fastening artificial objects, such as heart valves or implants, for example.

### BACKGROUND OF THE INVENTION

FIG. 1A illustrates a portion of the heart 12, the mitral valve 18, and the left ventricle 14. The mitral valve is at its boundary circumferenced by an annulus 20. The valve has two cusps or leaflets 22, 24. Each of these cusps or leaflets 22, 24 are connected to a respective papillary muscle 27, 29 via their respective connecting chordae 26, 28. In normal healthy individuals the free edges of the opposing leaflets will close the valve by coaptation. However, for some individuals the closure is not complete, which results in a regurgitation, also called valvular insufficiency, i.e. back flow of blood to the left atrium making the heart less effective and with potentially severe consequences for the patient. FIG. 1B illustrates a mitral valve 18, in which the leaflets 22, 24 do not close properly. This commonly occurs when the annulus 20 becomes dilated. One surgical procedure to correct this is to remove a portion of the leaflet 24 and stitch the cut edges together with one another. The procedure will pull back the annulus 20 to a more normal position. However, the strength of the leaflet 24 is altered. Similar problems with a less effective heart function occur if one or both leaflets are perforated to such an extent that blood is flowing towards the left atrium, although the leaflets close properly.

In some conditions of degenerated heart function, the leaflets do not present a solid surface, as in a degenerative valve disease. The leaflet may also be ruptured, most commonly at an edge of a leaflet, resulting in an incomplete coaptation. Hence, cardiac devices and methods are developed for repairing of one or more leaflets of a heart valve, or other related anatomical structures, such as the chordae attached to the ventricular side of leaflets.

FIG. 2A and 2B illustrate a prior art cardiac implant device and method for repairing of one or more leaflets of a heart valve as is described in the applicant's previous EP-patent (EP 1 853 199 B1), where the device 40 comprises a first and a second loop-shaped support 42, 44, which are connected to each other by means of a connecting part 48 so as to form a coil-shape. The coil-shape of the device is advantageous during insertion, since the device 40 may then be rotated into position, as described in the patent in more details. One of the supports 44 may be open, e.g. C or D or any other anatomical shaped such that the support 44 presents an end to lead the movement of the support 44 when being rotated into position. The position of the supports 42, 44 are fastened by fasteners 56, which are inserted and fastened by hand or small screwdriver.

It is found that the prior art cardiac implant devices, such as depicted above, work very well, but there are still some disadvantages relating to the fastening of the cardiac device into the annulus of the heart valve. The cardiac devices, such as other object, like cardiac implant or tissue or skin transplant are typically manually sutured by a traditional needle and yarns, or by using different kinds of mechanical screws.

One major issue in the fastening is to fastene the object firmly but at the same time so that it does not cause enormous stress or breakage to the tissue to which the object is fastened. For example, if the object to be fastened must be in a compressed state after fastening, the suture or screw must be tightened firmly, which is very challenging task to do so that the tissue to which the object is fastened is not compressed too much and thereby no damages are caused to the tissue. This may occur for example when the double ring cardiac implant device 40 having the first and second loop-shaped supports 42, 44 must be pressed firmly against each other so that the tissue 20 will be fastened tightly between the first and second loop-shaped supports 42, 44. In some cases there might be even a need for fastening the object so that it will be in a decompressed state after fastening, which is very challenging or even impossible task with the known prior art solutions.

### SUMMARY OF THE INVENTION

An object of the invention is to alleviate and eliminate the problems relating to the known prior art. Especially the object of the invention is to provide a medical fastening device for fastening an object to a tissue so that the object can be fastened and compressed tightly and firmly to the tissue, or decompressed so that the tissue is not stressed by the same compression force as the object. The object may be for example an implant, such as a cardiac implant, or other tissue or skin transplant.

The object of the invention can be achieved by the features of independent claims.

The invention relates to a medical fastening device according to claim 1.

According to an embodiment of the invention a medical fastening or fastening device for fastening or coupling an object into a tissue comprises a helical body structure and proximal and distal portions. The helical body structure comprises a thread or is arranged in a form of a thread for example after the helical body structure is introduced with the tissue. The inner portion of the helical body structure is advantageously a hollow or an empty space so to receive the portion of the object and/or tissue. The thread has a lead and a pitch or other property between the proximal and distal portions. In addition, the helical body structure comprises a first portion for example in the proximal or distal portion of the medical fastening device and a second portion next to said first portion.

The property of the thread is advantageously a pitch or lead of the thread in a direction of the longitudinal axis of the helical body structure. The lead is a distance along the longitudinal axis of the helical body structure that is covered by one complete rotation of the medical fastening device)(360°). The pitch is the distance from the crest or ridge of one thread to the next, which is the same as lead when the thread of the medical fastening device has a one start, but differs if there are two or more starts. In general, lead is equal to pitch times the number of starts.

The property of the thread may also be a helix angle (opening angle) or diameter of the thread or the pitch (so the material of the helical body structure) or diameter of the helical body structure as such. However, these are only examples and the invention is not limited to those only. For example, when the diameter of the lead or the helical body structure varies, also the area of the lead or helical body structure varies and thus the pressure induced by the structure varies or the pressure can be spread differently, which might be advantageous in some applications. As an example, the diameter of the helical body structure may be larger in the second portion than in the first or third portions, whereupon the helical body structure may force the object and/or tissue at the second portion to expand larger. Also the pressure can be spread to a larger area at the second portion by this embodiment. Alternatively, the diameter of the helical body structure may be smaller in the second portion than in the first or third portions, whereupon the helical body structure may force the object and/or tissue at the second portion to contract smaller. In this embodiment the pressure can be spread to a larger area at the first and/or in the third portions.

According to an advantageous embodiment the thread of the helical body structure is arranged to extend through the first and second portions. In addition the properties, such as a pitch or lead for example, of the thread is arranged to vary along the longitudinal axis of the helical body structure so that the property of the thread has a first value in the first portion and a second value in the second portion. By this a different compressive or decompressive force can be provided to the object and/or tissue introduced in the first and second portions of the helical body structure. The first value advantageously differs from the second value and the property of the thread can vary in a linear or in a non-linear way in the direction of the longitudinal axis of the medical fastening device, depending on the application where the medical fastening device is used.

According to an embodiment the medical fastening device may also comprise at least two helical body structures having a common longitudinal axis. In this embodiment both helical body structures comprise own thread having a lead between the proximal and distal portions of the helical body structures. Each helical body structure comprises also a first portion for example in the proximal portion and a second portion next to said first portion. Also threads of the helical body structures extend through the first and second portions. The properties of the threads vary advantageously along the helical body structure so that the property of the thread in the both helical body structures has a first value in the first portion and a second value in the second portion thereby providing different compressive or decompressive force to the object and/or tissue introduced in said first and second portions of the helical body structure analogously as in the embodiment with the one helical body structure. However, when the two or more helical body structures are used the compressive or decompressive force induced into the object and/or to the tissue can be more stronger and in addition the object can be caught and fastened better also in lateral direction (perpendicular to the longitudinal axis of the helical body structures).

According to an embodiment the helical body structure may also comprise a third portion next to the second portion. The third portion may be arranged for example in the distal portion of the helical body structure so that the second portion locates between the first and the third portions. The thread of the helical body structure extends advantageously along the helical body structure through said first, second and third portions. In addition the properties of the thread, such as for example a pitch and lead, is arranged to vary along the helical body structure so that the property of the thread has a first value in the first portion and a second value in the second portion and the third value in the third portion. Depending on the application the first value differs from the second value, and again the first value and the third value may be the same or different with each other.

According to an advantageous embodiment the pitch and/or lead of the thread is shorter (or has a greater dense) in the second portion than in the first and/or third portion. By this the object can be arranged in a compressed state after fastening which might be useful in some situation, for example when the object must be compressed more than the tissue around it or the object is another tissue which is tougher that the tissue to which said tougher tissue is fastened. For example the first and second loop-shaped supports 42, 44 of the double ring cardiac implant device 40 (shown in FIG. 2A) can be pressed by this way firmly against each other so that the tissue 20 will be fastened tightly between the first and second loop-shaped supports 42, 44.

According to another advantageous embodiment the pitch and/or lead of the thread is longer (or has a smaller dense) in the second portion than in the first and/or third portion. By this the object can be arranged in a decompressed state after fastening which might be useful in some situation, for example when the object must not be compressed as much as the tissue around it or the object is another tissue which is softer that the tissue to which said softer tissue is fastened. For example the first and second loop-shaped supports 42, 44 of the double ring cardiac implant device 40 (shown in FIG. 2A) can be unpressed or decompressed by this way farther from each other so that the tissue 20 will not be pressed or compressed as much between the first and second loop-shaped supports 42, 44 than in a steady state (no external forces applied to the loop-shaped supports).

According to an embodiment the start of the helical body structures in the distal end, such as the third portion, may have a bigger lead and/or pitch than the second portion, which advantageously together with the object forces the threads of the second portion to “open” or stretch in the longitudinal direction of the helical body structure at least little bit when the helical body structures is introduced during fastening with the object, whereupon when the distal end or the third portion has overpassed the object, threads of the of the second portion will compress back to the original state. In this way the object can be compressed by the second portion of the helical body structures. According to another embodiment the start of the helical body structures in the distal end, such as the third portion, may have a smaller lead and/or pitch than the second portion, whereupon the effect is opposite and the outcome is that the threads of the of the second portion will “open” or unfold back to the original state (decompress).

It is to be noted that the object might be a cardiac implant, such as for example annuloplasty ring, but also a tissue, implant or skin transplant, whereupon also the tissue, implant or skin transplant can cause the second portion to “open” or stretch or to behave opposite such as described above.

As can be noted in this document the property of the thread can have different forms and it can be implemented or achieved in numerous ways, such as the thread may have for example a certain pitch and/or lead before introducing to the object and/or the tissue. According to an embodiment the helical body structure may also comprise shape memory material, such as Nitinol comprising nickel and titanium, being resiliently biased to change the value of the property of the threads advantageously after the introduction with the tissue. For example property of the threads in first, second and /or third portions can be changed due to shape memory material in these portions so that a certain compressive or decompressive force can be applied to the object and/or tissue in said portion of the helical body structure in question after introducing said portion to the tissue, for example. According to an example the helical body structure may comprise shape memory material resiliently biased so to form a thread with certain property after introduced with the tissue.

Furthermore, according to an embodiment the distal portion of the helical body structure may comprise a non-traumatic tip end, which dilates tissue fibres when introduced with the tissue and thereby both helps to introduce and penetrate the distal portion to the tissue but at the same time also to catch the object or twist around the object. In addition, the proximal portion of the helical body structure comprises advantageously a grip portion via which the medical fastening device can be rotated around its longitudinal axis by an introduction device. The grip portion may be implemented either a certain protrusion or projection portion or a bend portion, such as 90° angle portion.

The medical fastening device comprises advantageously also a securing member for engaging and securing the medical fastening device with the object in a direction of the longitudinal axis of the helical body structure and a lateral direction being perpendicular to the longitudinal axis. In this way the medical fastening device prevents the object to slip away from the hollow inner space or in other words prevents the medical fastening device to rotate and open itself, but also prevents the medical fastening device to be tightened too much or “over” the object to be fastened so that the object would be slip out and away via the proximal end portion of the medical fastening device.

According to an example the securing member comprises a slot or a concave portion having an opening so to receive the object into the opening. When the medical fastening device is screwed or turned during fastening, the object will be received into the hollow inner space, and until the object reaches the opening, the slot or a concave portion of the securing member of the medical fastening device will snap over the object thereby securing and locking the medical fastening device to the object. Advantageously the slot or a concave portion with the opening is arranged in the proximal portion of the medical fastening device.

According to an example the grip portion may function as a stopping member to prevent the medical fastening device to be fastened too tight or “turned over” the object and thereby to prevent the object to slip away from the hollow inner space via the end of the proximal portion of the medical fastening device. The grip portion functioning as the stopping member is advantageously arranged to elongate essentially over the hollow inner space and in addition it is advantageously arranged to extend over the hollow inner space essentially perpendicular to the longitudinal axis. Furthermore the grip portion functioning as the stopping member is advantageously arranged in the proximal portion of the medical fastening device.

Advantageously the securing member of the medical fastening device comprises both the slot or concave portion with the opening and the stopping member. In this case the opening and the stopping member are arranged so that there is distance between them so to arrange space enough for the object to be secured.

According to an embodiment the helical body structure may also comprises shape memory material, which is advantageously resiliently biased to form the securing member after introduced with the tissue. The shape memory material may be resiliently biased to form especially the opening and/or the grip or stopping member, when the portion of the helical body structure comprising said shape memory material is contacted with the tissue.

The securing member offers advantages, such as securing the medical fastening device to the object so that the loosening or unwanted turning of the medical fastening device can be avoided. In addition also fastening the medical fastening device too tight and even over or by the object or a target depth can be avoided.

The present invention offers advantages over the known prior art, such as fastening an object to a tissue so that the object can be fastened and compressed or decompressed tightly and firmly to the tissue, so that the tissue is not stressed by the same compression or decompression force as the object. This thus minimizes probability to cause breakages or damages to the tissue due to the tightly and firmly fastening. In addition the present invention also enables to fastene the object so that the object or the tissue is in a decompressed or slightly opened state after fastening.

The exemplary embodiments presented in this text are not to be interpreted to pose limitations to the applicability of the appended claims. The verb “to comprise” is used in this text as an open limitation that does not exclude the existence of also unrecited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

### BRIEF DESCRIPTION OF THE DRAWINGS

Next the invention will be described in greater detail with reference to exemplary embodiments in accordance with the accompanying drawings, in which:

FIGS. 1A-1B illustrate schematically a portion of a heart and mitral valve,

FIGS. 2A-2B illustrate a prior art cardiac implant device for repairing of one or more leaflets of a heart valve, and

FIG. 3 illustrates a principle of an exemplary medical fastening device for fastening an object into a tissue according to an advantageous embodiment of the invention,

FIGS. 4-5 illustrate an exemplary medical fastening device with two helical body structures according to an advantageous embodiment of the invention,

FIGS. 6-7 illustrate an exemplary medical fastening device with a securing member according to an advantageous embodiment of the invention, and

FIGS. 8-9 illustrates an exemplary medical fastening device in use according to an advantageous embodiment of the invention.

### DETAILED DESCRIPTION

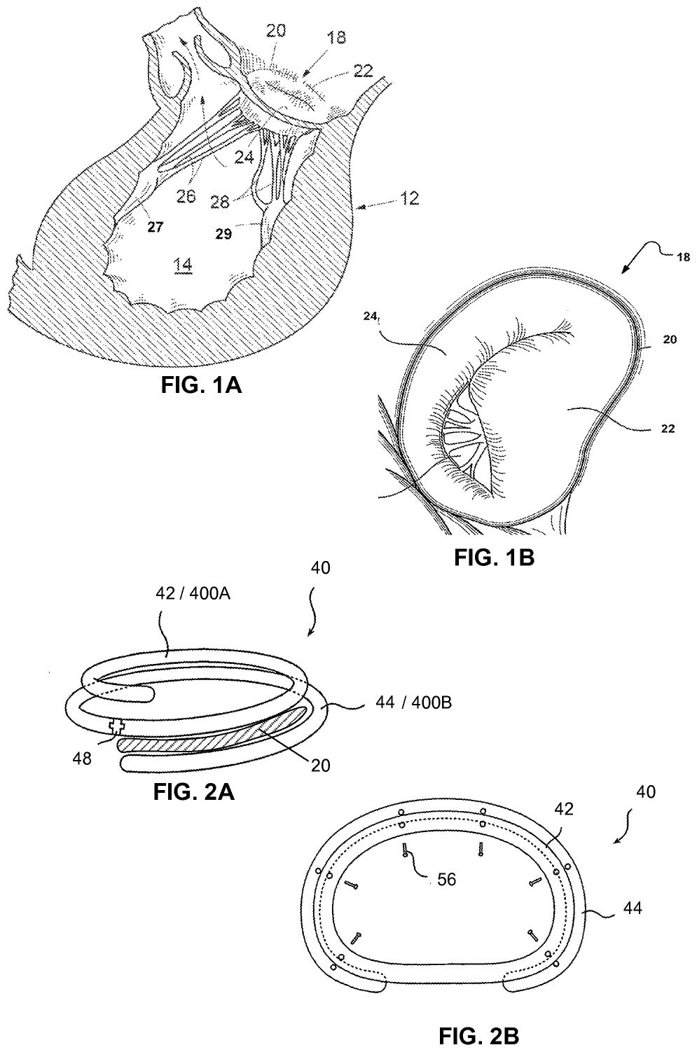

FIGS. 1A-1B and 2A-2B are already discussed in more details in connection with the background of the invention portion above.

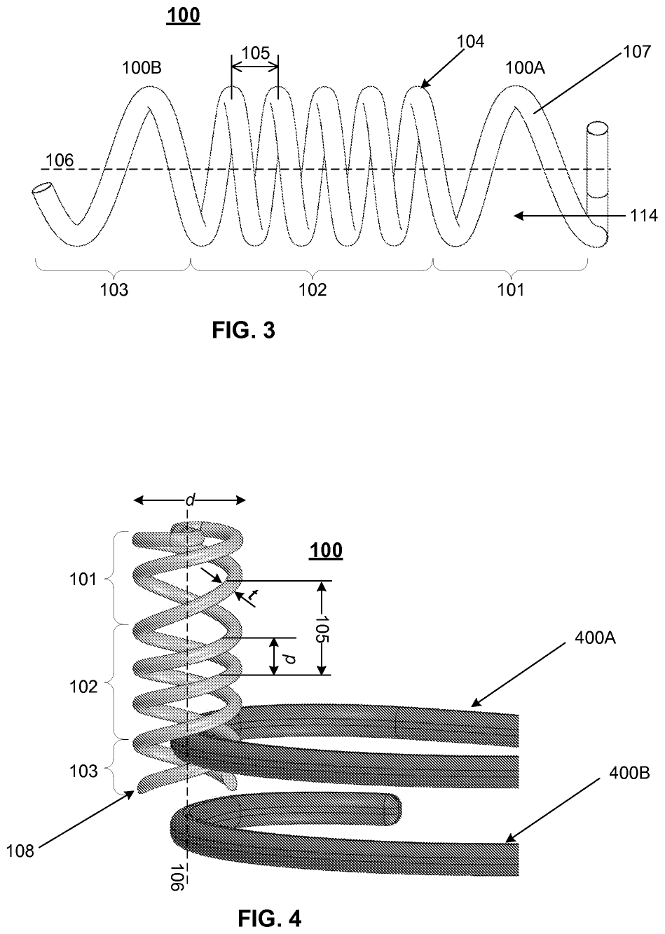

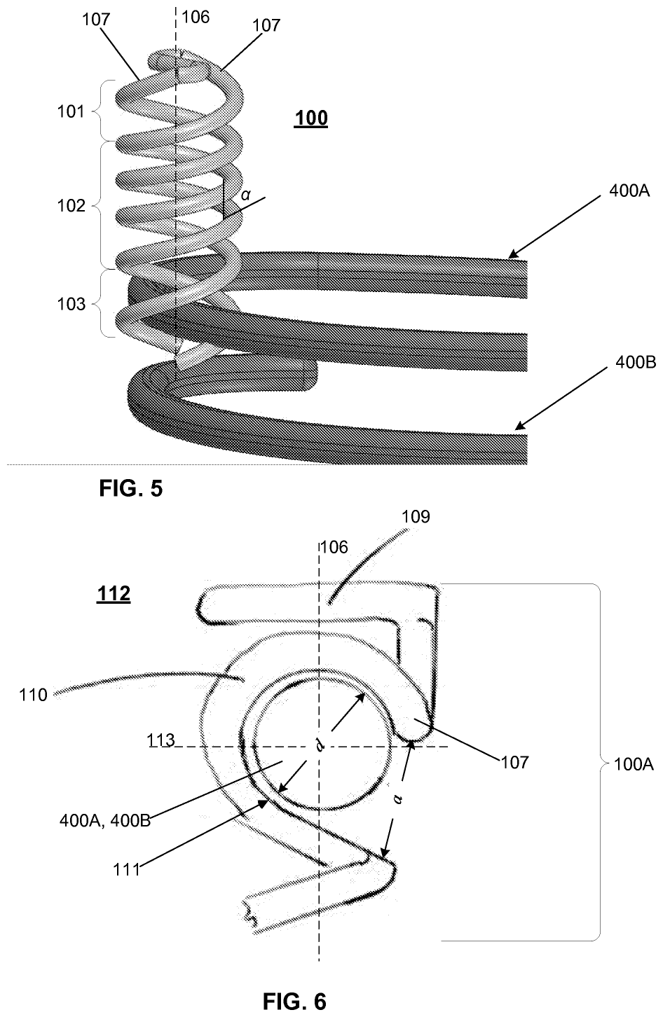

FIG. 3 illustrates a principle of an exemplary medical fastening device 100 for fastening or coupling an object 400A, 400B into a tissue 20 according to an advantageous embodiment of the invention, which comprises a helical body structure 107 and proximal 100A and distal 100B portions. FIGS. 4-5 illustrate an exemplary medical fastening device 100 with two helical body structures 107.

In both examples the helical body structure comprises or is in a form of a thread 104, where the inner portion surrounded by the thread 104 is an empty space or hollow 114 so to receive at least part of the object and/or the tissue. In the examples illustrated the thread 104 has a lead 105 and a pitch p between the proximal and distal portions in a direction of the longitudinal axis 106 of the helical body structure 107, but can naturally have also other property described in this document, such as helix angle or opening angle a, diameter t of the thread or the pitch (so the diameter t of the material of the helical body structure) or diameter d of the helical body structure as such. In addition, the helical body structure comprises a first portion 101 advantageously in the proximal 100A portion of the medical fastening device and a second portion 102 next to said first portion 101.

The lead 150 is a distance along the longitudinal axis 106 of the helical body structure 107 that is covered by one complete rotation of the medical fastening device 360°. The pitch p is the distance from the crest or ridge of one thread to the next, which is the same as lead 105 when the thread of the medical fastening device has a one start, but differs if there are two or more starts (as is the case in FIGS. 4 and 5, for example).

As can be seen in FIGS. 3-5 that the thread of the helical body structure 107 is arranged to extend through the first 101 and second 102 portions. In addition the properties, such as a lead 105 or pitch p for example, of the thread is arranged to vary along the longitudinal axis 106 of the helical body structure 107 so that the property of the thread has a first value in the first portion 101 and a second different value in the second portion 102. Even if not shown in Figures the diameter d of the helical body structure may be larger in the second portion 102 than in the first 101 (or in the third 103) portion(s), whereupon the helical body structure may force the object 400A, 400B and/or tissue 20 at the second portion 102 to expand for example perpendicular to the longitudinal axis 106. Alternatively, the diameter d of the helical body structure may be smaller in the second portion 102 than in the first 101 (and/or in the third 103) portion(s), whereupon the helical body structure may forces the object 400A, 400B and/or tissue 20 at the second portion 20 to contract essentially towards the longitudinal axis 106.

As can be seen in FIGS. 4-5, the medical fastening device 100 has two helical body structures 107 with a common longitudinal axis 106. In this embodiment both helical body structures comprise own thread 104 having a lead 105 and pitch p between the proximal and distal portions of the helical body structures. Each helical body structure 107 comprises also a first portion 101 for example in the proximal portion and a second portion 102 next to said first portion 101.

Further the helical body structure 107 may also comprise a third portion 103 next to the second 102 portion, as can be seen in FIGS. 3-5. The third portion 103 is advantageously arranged in the distal portion 100B of the helical body structure 107 so that the second portion 102 locates between the first and the third portions 101, 103. The thread 104 of the helical body structure extends advantageously along the helical body structure through said first 101, second 102 and third 103 portions. In addition the properties of the thread, such as for example a pitch and lead 105, is arranged to vary along the helical body structure so that the property of the thread 104 has a first value in the first portion 101 and a second value in the second portion 102 and the third value in the third portion 103.

In addition, as can be seen in FIGS. 3-5 the lead 105 and/or pitch p of the thread 104 is shorter (or has a greater dense) in the second portion 102 than in the first portion 101. By this the object 400A, 400B can be arranged in a compressed state after fastening. Even if not illustrated the pitch p and/or lead 105 of the thread 104 may also be longer (or has a smaller dense) in the second portion 102 than in the first portion 101, whereupon a decompressed state after fastening for the object can be achieved.

Furthermore, the distal portion 100B of the helical body structure 107 may comprise a tip end 108, which both helps to introduce and penetrate the distal portion to the tissue 20 but at the same time also to catch the object 400A, 400B or twist around the object 400A, 400B. In addition the proximal portion of the helical body structure 107 may advantageously comprise a grip portion 109 via which the medical fastening device 100 can be rotated around its longitudinal axis 106 by an introduction device (not shown), as can be seen for example in FIG. 6. The grip portion 109 may be implemented either a certain protrusion or projection portion or a bend portion, such as 90° angle portion.

FIG. 6 illustrates also a principle of an exemplary medical fastening device 100 with a securing member 112 for locking or securing the medical fastening device 100 to the object 400A, 400B after the object 400A, 400B is fastened to the tissue 20. The securing member 112 may e.g. comprise a slot 110 or a concave portion 110 having an opening 111, which advantageously locates at the proximal portion 100A of the medical fastening device 100. In the use the object 400A, 400B to be fastened is received into the opening 111 so that the opening will surround the object at least partially. The slot 110 or a concave portion may have a spring-like feature so to “snap” around the object, especially around the ring-type object (e.g. the cardiac implant device with the first and second loop-shaped supports 42, 44,) when screwed or turned around its longitudinal axis 106 and when the portion of the object locates “inside” the threaded helical body structure 107. In addition also the object 400A, 400B may be compressed slightly by the helical body structure 107 before snapping into the slot or the concave portion 110. The opening 111 is advantageously arranged in the proximal portion 100A of the medical fastening device 100.

When the medical fastening device 100 is screwed or turned during fastening, the portion of the object will be received into the hollow inner space 114, and when the object reaches the opening 111, the slot 110 or a concave portion 110 of the securing member 112 will snap over the object thereby encaging, securing and locking the medical fastening device 100 to the object 400A, 400B. The slot or a concave portion 110 may have a spring-like feature so that it bends when it is turned and faced against the object and will return to the normal state after the portion of the object is introduced to the opening 111. In addition, as can be seen in particularly in FIG. 6, a width a of the jaw or mouth of the opening 111 is advantageously arranged so that it is little bit smaller than the diameter or thickness d of the object 400A, 400B, whereupon the object 400A, 400B forces the slot or a concave portion 110 little bit the bend so that the object 400A, 400B can enter to the opening. Furthermore the extent or depth of the opening 111 must be big enough so to enable to receive the object 400A, 400B inside.

It is to be noted that the grip portion 109 may advantageously function also as a stopping member 109, as can be seen at least in FIGS. 6 and 8-9.

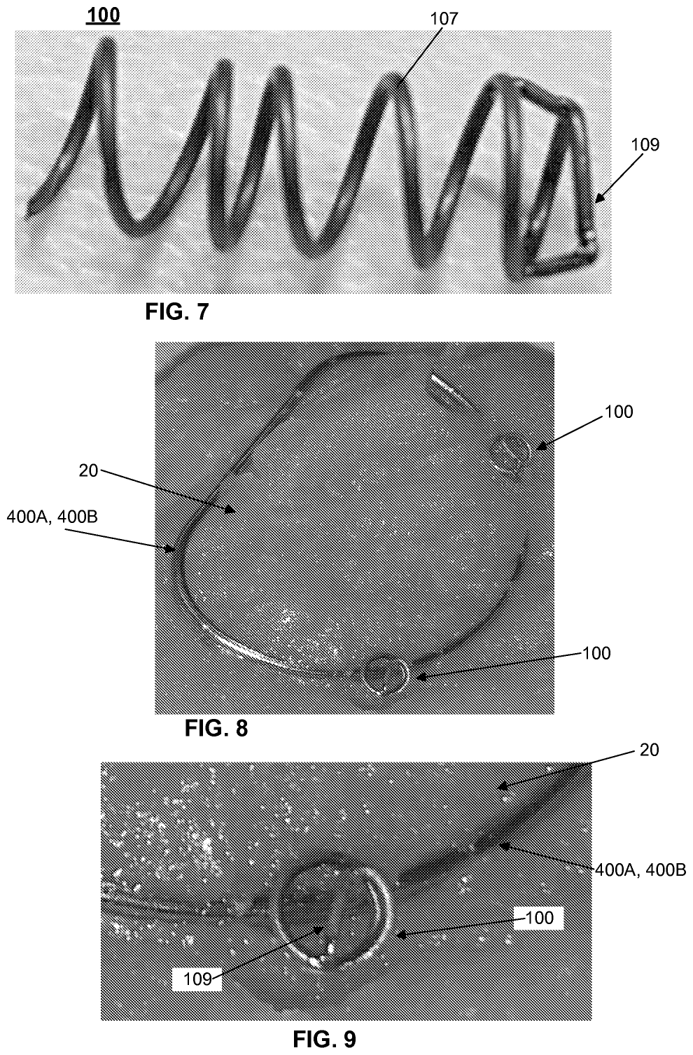

The grip portion or stopping member 109 may elongate e.g. over the hollow inner space 114 essentially perpendicular to the longitudinal axis 106, as can be seen e.g. in FIGS. 7-9, thereby preventing the medical fastening device 100 to be fastened too tight or “turned over” the object. Also the grip portion or stopping member 109 is advantageously arranged in the proximal portion 100A of the medical fastening device 100.

Overall the securing member 112 secures the medical fastening device 100 to the object and vice versa in a direction of the longitudinal axis 106 of the helical body structure 107, but also in a lateral direction 113 being perpendicular to the longitudinal axis 106. In this way the securing member 112 prevents the object 400A, 400B to slip away from the hollow inner space 114 and also the medical fastening device 100 to rotate and open by itself. In addition the securing member 112 also prevents the medical fastening device 100 to be tightened too much or “over” the object to be fastened so that the object would be slip out and away via the proximal end portion 101A of the medical fastening device 100.

FIGS. 8-9 illustrates an exemplary medical fastening device in use according to an advantageous embodiment of the invention, where the object 400A, 400B is fastened by the medical fastening device 100 to the tissue 20. As can be seen in FIGS. 8-9 the medical fastening device 100 is screwed around the object 400A, 400B so that the portion of the object locates inside the hollow inner space 114 of the medical fastening device 100. At the same time the grip portion or stopping member 109 prevents further screwing the medical fastening device 100 and thus also prevent the object 400A, 400B to slip out and away from the hollow inner space 101 of the medical fastening device 100 in the longitudinal direction 106. In addition the opening 111 (not shown in FIGS. 8-9) prevents the object 400A, 400B to slip out and away from the hollow inner space 114 of the medical fastening device 100 in the lateral direction 113.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

The features recited in dependent claims are mutually freely combinable unless otherwise explicitly stated.

1. A medical fastening device (100) for fastening an object (400A, 400B) into a tissue (20),
wherein:
the medical fastening device (100) having proximal (100A) and distal (100B) portions comprises at least two helical body structures (107) having a common longitudinal axis (106), where each of the helical body structure comprises own thread (104) having a lead (105) between said proximal and distal portions of the helical body structures,
each of said helical body structure comprises a first portion (101/103) and a second portion (102) next to said first portion (101), wherein
the threads of the helical body structures extend through said first (101) and second (102) portions and wherein at least one property of the thread varies along the longitudinal axis (106) of the helical body structure after introducing the helical body structure with the tissue (20) so that the property of the thread (104) in the both helical body structures has a first value in the first portion (101) and a second value in the second portion (102) so to provide different compressive force to the object and/or tissue introduced in said first and second portions of the helical body structure, and wherein the first value differs from the second value.

2. A device of claim 1, wherein the device comprises
a third portion (103) next to the second (102) portion so that the second portion (102) is between the first and the third portions (101, 103), and
the thread (104) of the helical body structure extends along the helical body structure through said first (101), second (102) and third (103) portions and wherein at least one property of the thread varies along the helical body structure after introducing the helical body structure with the tissue (20) so that the property of the thread (104) has a first value in the first portion (101) and a second value in the second portion (102) and the third value in the third portion (103), and wherein the first and/or third values differs from the second value.

3. A device of claim 2, wherein the first value and the third value are same; or wherein the first value and the third value differ from each other.

4. A device of claim 1, wherein the property of the thread (104) is a pitch and/or lead (105) of the thread in a direction of the longitudinal axis (106) of the helical body structure (107), helix angle or thickness of the helical body structure or diameter of the helical body structure (107), or width or depth of the thread.

5. A device of claim 1, wherein the pitch and/or lead of the thread (104) is shorter in the second portion (102) than in the first portion (101); or where the pitch and/or lead of the thread (104) is longer in the second portion (102) than in the first portion (101).

6. A device of claim 1, wherein the helical body structure (107) comprises shape memory material being resiliently biased to change the value of the property of the threads, such as distance between the adjacent pitches and/or leads, in said first portion (101) and/or in the second portion (102) after introducing with the tissue and thereby to provide different compressive force to the object and/or tissue introduced in said first portion (101) than in said second portion (102) of the helical body structure.

7. A device of claim 1, wherein the distal portion (100B) of the helical body structure (107) comprises a non-traumatic tip end (108) to be introduced to the tissue (20).

8. A device of claim 1, wherein the proximal portion (100A) of the helical body structure (107) comprises a grip portion (109) to be engaged with an introduction device and for rotating the medical fastening device (100) around its longitudinal axis (106).

9. A device of claim 1, wherein fastening device (100) comprises a securing member (112) for engaging and securing the medical fastening device (100) with the object (400A, 400B) in a direction of the longitudinal axis (113) of the helical body structure (107) and a lateral direction (103) being perpendicular to the longitudinal axis (106), and thereby to prevent the object (400A, 400B) and the medical fastening device (100) to separate from each other.

10. A device of claim 9, wherein the securing member (112) comprises a slot (110) or a concave portion (110) having an opening (111) and arranged in the proximal portion (100A) of the medical fastening device (100) so to receive the object (400A, 400B) to be fastened into an opening (111) and for locking the medical fastening device (100) to the object (400A, 400B) after the object (400A, 400B) is fastened to the tissue (20) by the medical fastening device (100).

11. A device of any of claim 9, wherein the securing member (102) comprises a grip portion functioning as a stopping member (109) arranged to elongate essentially over a hollow inner space (101) of the spring-like helical body structure (107) and thereby configured to prevent the object to slip away from the hollow inner space via the end of the proximal portion (100A).

12. (canceled)

13. (canceled)
